(19) United States
(12) Patent Application Publication
(10) Pub. No.: US 2009/0304661 A1
(43) Pub. Date: Dec. 10, 2009

(54) Method And Composition For Proctecting Neuronal Tissue From Damage Induced By Elevated Glutamate Levels

(75) Inventors: Vivian I. Teichberg, Savyon (IL); Alexander Zlotnik, Beer-Sheva (IL); Yoram Shapira, Omer (IL)

Correspondence Address: MARTIN D. MOYNIHAN d/b/a PRTSI, INC., P.O. BOX 16446, ARLINGTON, VA, 22215, US

(73) Assignees: Yeda Research And Development Co. Ltd. at the Weizmann Institute of Sceince, Rechovot (IL); Mor Research Applications Ltd., Tel-Aviv (IL)

(21) Appl. No.: 12/225,105

(22) PCT Filed: Mar. 8, 2007
(86) PCT No.: PCT/IL2007/000297

§ 371 Date: Aug. 5, 2009

Related U.S. Application Data
(60) Provisional application No. 60/782,516, filed on Mar. 16, 2006.

(51) Int. Cl. A61K 45/00; A61K 38/43; A61K 38/45; A61K 38/44; A61P 25/00

(52) U.S. Cl. 424/93.73; 424/94.1; 424/94.5; 424/94.4

(57) ABSTRACT

A method of reducing extracellular brain glutamate levels is provided. The method comprising administering to a subject in need thereof an agent capable of modulating stress hormone activity thereby reducing blood glutamate levels, thereby reducing extracellular brain glutamate levels.

### FIELD AND BACKGROUND OF THE INVENTION

The present invention relates to a method and composition for protecting the central nervous system (CNS) from damage induced by abnormal levels of glutamate, which may result from, for example, a stroke.

The central nervous system is composed of trillions of nerve cells (neurons) that form networks capable of performing exceedingly complex functions.

The amino acid L-glutamic acid (Glutamate), mediates many of the excitatory transactions between neurons in the central nervous system. Under normal conditions, accumulation of glutamate in the extracellular space is prevented by the operation of a recycling mechanism that serves to maintain neuronal glutamate levels despite continual loss through transmitter release (Van der Berg and Garfinkel, 1971; Kennedy et al., 1974). Glutamate, released by glutamatergic neurons, is taken up into glial cells where it is converted into glutamine by the enzyme glutamine synthetase. Glutamine reenters the neurons and is hydrolyzed by glutaminase to form glutamate, thus replenishing the neurotransmitter pool.

This biochemical pathway also serves as an endogenous neuroprotective mechanism, which functions by removing the synaptically released glutamate from the extracellular space and converting it to the nontoxic amino acid glutamine before toxicity occurs. The removal of glutamate from the extracellular space into brain takes place via specific glutamate transporters that co-transport glutamate and sodium ions. The driving force for this co-transport resides in the concentration gradient between the high extracellular and low intracellular concentrations of sodium ions. The excitotoxic potential of glutamate (i.e., defined as the ability of excess glutamate to overexcite neurons and cause their death) is held in check as long as the transport process is functioning properly. However, failure or reduction in the transport process such as under ischemic conditions, results in accumulation of glutamate in the extracellular synaptic fluid and excessive stimulation of excitatory receptors, a situation that leads to neuronal death.

Two additional factors complicate and make matters worse: (i) overstimulated neurons begin to release excessive quantities of glutamate at additional synaptic junctions; this causes even more neurons to become overstimulated, drawing them into a neurotoxic cascade that reaches beyond the initial zone of ischemia; and, (ii) overstimulated neurons begin utilizing any available supplies of glucose or oxygen even faster than normal, which leads to accelerated depletion of these limited energy resources and further impairment of the glutamate transport process. This biochemical cascade of induction and progression may continue for hours or days and causes delayed neuronal death.

Abnormally high glutamate (Glutamate) levels in brain interstitial and cerebrospinal fluids are the hallmark of several neurodegenerative conditions. These include acute brain anoxia/ischemia i.e stroke (Graham et al., 1993; Castillo et al., 1996), perinatal brain damage (Hagberg et al., 1993; Johnston, 1997), traumatic brain injury (Baker et al., 1993; Zauner et al., 1996), bacterial meningitis (Spranger et al, 1996), subarachnoid hemorrhage, open heart and aneurysm surgery (Persson et al., 1996; Saveland et al., 1996), hemorrhagic shock (mongan et al. 1999, 2001), newly diagnosed epilepsy (Kalviainen et al., 1993), acute liver failure (Rose et al. 2000) and various chronic neurodegenerative diseases such as glaucoma (Dreyer et al., 1996), amyotrophic lateral sclerosis (Rothstein et al., 1990; Shaw et al., 1995), HIV dementia (Ferrarese et al. 2001) and Alzheimer's disease (Pomara et al., 1992).

Thus, one object of medical therapy is to break or eliminate the above described cascade process and thus prevent glutamate associated neuronal damage.

Since glutamate excitotoxicity is mediated by the glutamate receptors, a potential therapeutic approach has been to develop and apply various selective glutamate receptor antagonists in animal models of neurodegeneration. Though displaying powerful neuroprotective effects in experimental stroke and head trauma, the glutamate receptor antagonists failed in clinical trials mainly because of their adverse or even lethal effects (Birmingham, 2002; Lutsep and Clark, 2001; Palmer, 2001).

Attempts have also been made to increase the activity of the various glutamate transporters, present on glia and neurons, which take up Glutamate from the extraneuronal fluid and thereby limiting glutamate excitatory action and excitotoxicity. However, none of the above-described approaches have been successful in providing a viable therapeutic approach for lowering glutamate levels.

In light of these failures and the need of alternative approaches to the treatment of neurodegenerative disorders involving glutamate excitotoxicity, the present inventors has hypothesized that excess glutamate in brain interstitial (ISF) and cerebrospinal (CSF) fluids could be eliminated by increasing the relatively poorly studied brain to blood glutamate efflux mechanism. Increasing the efflux can be achieved by lowering the glutamate levels in blood thereby increasing glutamate transport from brain ISF/CSF to blood.

The present inventor has previously uncovered that by maximally activating two enzymes, glutamate-pyruvate transaminase (GPT) and glutamate-oxaloacetate transaminase (GOT), glutamate degradation in the blood is increased (PCT IL03/00634 to the present inventor). These two enzymes are two examples of a wider group of enzymes that use glutamate as a substrate in the general equation

A+GLUTAMATE←(enzyme)→C+D

whereby A represents the co-substrate. ←(enzyme)→ symbolizes a reversable enzyme and C and D are metabolites of the enzyme. For example

Glutamate+oxaloacetate←(GOT)→2-keto-glutarate+aspartate

Another example is:

Glutamate+pyruvate←(GPT)→2-keto-glutarate+alanine

A third example is:

Glutamate+4-methyl-2-oxopentoate←(branched-chain-amino-acid transaminase)→2-ketoglutarate+Valine.

Examples for different substrates that work on the same enzyme are:

Glutamate+2-oxohexanedioic acid←(GOT)→2-keto-glutarate+2-aminohexanedioic acid.

Glutamate+2-oxo-3-phenylpropionic acid←(GOT)→2-keto-glutarate+phenylalanine.

Glutamate+3-hydroxy-2-oxopropionic acid←(GOT)→2-keto-glutarate+serine.

Glutamate+5-oxopentanoate←(GPT)→2-keto-glutarate+5-aminopentanoate

Glutamate+4-oxobutanoate←(GPT)→2-keto-glutarate+4-aminobutanoate

Glutamate+glyoxalate←(GPT)→2-keto-glutarate+glycine

Another common feature that these enzymes share is that they use pyridoxal phosphate as a cofactor.

As stated, these enzymes reversibly convert glutamate into 2-keto glutarate. This causes blood glutamate levels to further decrease, below basal levels thereby creating a far steeper gradient of glutamate levels between the brain ISF/CSF and blood, than normally exists. In order to reach a novel equilibrium, glutamate is transported from the brain to the blood thus lowering the elevated levels of glutamate in the brain. As long as the glutamate levels are low in the blood, this brain to blood efflux will continue. In order to keep GOT and GPT working at their maximum levels for the conversion of glutamate into 2-ketoglutarate (Vₘₐₓ) their respective substrates, oxaloacetate and pyruvate have to be administered.

As stated above both glutamate-oxaloacetate transaminase and glutamate-pyruvate transaminase metabolize glutamate, while using oxaloacetate and pyruvate as their respective co-substrates. There are however many other transaminases in the body that can metabolize glutamate such as glutamate oxaloacetate transaminase, branched-chain-amino-acid transaminase, alanine transaminase, GABA aminotransferases and many others. For each enzyme according to its reaction, a specific substrate such as succinate semialdehyde for 4-aminobutyrate transaminase should be used.

Conversely, although pyruvate and oxaloacetate are possibly the best substrates for the glutamate transaminases other substrates such as 2-oxohexanedioic acid, 2-oxo-3-sulfopropionate, 2-oxo-3-sulfinopropionic acid, 2-oxo-3-phenylpropionic acid or 3-indole-2-oxopropionic acid instead of oxaloacetate and 5-oxopentanoate, 6-oxo-hexanoate or glyoxalate instead of pyruvate can be used.

The conversion of glutamate to 2-ketoglutarate is reversible. Thus, upon glutamate metabolization via an enzymatic reaction into 2-ketoglutarate, there is a buildup of 2-ketoglutarate which can cause the enzyme to work in the reverse direction and convert 2-ketoglutarate into glutamate. It is therefore beneficial to further break down 2-ketoglutarate and in this way insure the continual metabolism of glutamate. One such enzyme that metabolizes 2-ketoglutarate is 2-ketoglutarate dehydrogenase. The general reaction is:

2-ketoglutarate+lipoamide←(2-ketoglutarate dehydrogenase)→S-succinyldihydrolipoamide+CO₂.

While reducing the present invention to practice, the present inventor uncovered that stress conditions may also increase the efflux of excess Glu from the brain parenchyma into blood. These findings suggest the use of stress hormones for reducing extracellular brain glutamate levels. This efflux may be increased by the co-administration of previously described blood glutamate scavengers such as oxaloacetate.

### SUMMARY OF THE INVENTION

According to one aspect of the present invention there is provided a method of reducing extracellular brain glutamate levels, the method comprising administering to a subject in need thereof an agent capable of modulating stress hormone activity thereby reducing blood glutamate levels, thereby reducing extracellular brain glutamate levels.

According to another aspect of the present invention there is provided a pharmaceutical composition comprising as active ingredients at least two agents capable of reducing blood glutamate levels, wherein at least one of the at least two agents is capable of modulating stress hormone activity thereby reducing blood glutamate levels and a pharmaceutically acceptable carrier.

According to yet another aspect of the present invention there is provided an article of manufacture comprising packaging material and a pharmaceutical composition identified for reducing extracellular brain glutamate levels being contained within the packaging material, the pharmaceutical composition comprising, as an active ingredient, an agent capable of modulating stress hormone activity thereby reducing blood glutamate levels and a pharmaceutically acceptable carrier.

According to still another aspect of the present invention there is provided a method of reducing extracellular brain glutamate levels in a subject in need thereof, the method comprising: (a) obtaining a blood sample; (b) contacting the blood sample with an agent capable of modulating stress hormone activity thereby reducing glutamate levels of cells present in the blood sample to thereby obtain glutamate depleted blood cells; and (c) introducing the glutamate depleted blood cells into the subject, thereby reducing extracellular brain glutamate levels thereof.

According to further features in preferred embodiments of the invention described below, the agent capable of modulating stress hormone activity thereby reducing blood glutamate levels is a stress hormone agonist.

According to still further features in the described preferred embodiments the agent capable of modulating stress hormone activity thereby reducing blood glutamate levels is a stress hormone antagonist.

According to still further features in the described preferred embodiments the stress hormone agonist comprises an adrenergic receptor agonist.

According to still further features in the described preferred embodiments the adrenergic receptor agonist is an alpha 1 or alpha 2 agonist.

According to still further features in the described preferred embodiments the adrenergic receptor agonist is a beta 2 agonist.

According to still further features in the described preferred embodiments the stress hormone antagonist comprises is an adrenergic receptor antagonist.

According to still further features in the described preferred embodiments the adrenergic receptor antagonist is a beta 1 antagonist.

According to still further features in the described preferred embodiments the method further comprising administering an additional agent capable of reducing blood glutamate levels prior to, concomitant with or following administering the stress hormone.

According to still further features in the described preferred embodiments the method further comprising contacting the blood sample with an additional agent capable of reducing blood glutamate levels prior to, concomitant with or following step (b).

According to still further features in the described preferred embodiments the agent is at least one glutamate modifying enzyme and/or a modification thereof.

According to still further features in the described preferred embodiments the at least one glutamate modifying enzyme is selected from the group consisting of a transaminase, a dehydrogenase, a decarboxylase, a ligase, an aminomutase, a racemase and a transferase.

According to still further features in the described preferred embodiments the transaminase is selected from the group consisting of glutamate oxaloacetate transaminase, glutamate pyruvate transaminase, acetylornithine transaminase, ornithine-oxo-acid transaminase, succinyldiaminopimelate transaminase, 4-aminobutyrate transaminase, alanine transaminase, (s)-3-amino-2-methylpropionate transaminase, 4-hydroxyglutamate transaminase, diiodotyrosine transaminase, thyroid-hormone transaminase, tryptophan transaminase, diamine transaminase, cysteine transaminase, L-Lysine 6-transaminase, histidine transaminase, 2-aminoadipate transaminase, glycine transaminase, branched-chain-amino-acid transaminase, 5-aminovalerate transaminase, dihydroxyphenylalanine transaminase, tyrosine transaminase, phosphoserine transaminase, taurine transaminase, aromatic-amino-acid transaminase, aromatic-amino-acid-glyoxylate transaminase, leucine transaminase, 2-aminohexanoate transaminase, ornithine(lysine) transaminase, kynurenine-oxoglutarate transaminase, D-4-hydroxyphenylglycine transaminase, cysteine-conjugate transaminase, 2,5-diaminovalerate transaminase, histidinol-phosphate transaminase, diaminobutyrate-2-oxoglutarate transaminase, udp-2-acetamido-4-amino-2,4,6-trideoxyglucose transaminase and aspartate transaminase.

According to still further features in the described preferred embodiments the dehydrogenase is a glutamate dehydrogenase.

According to still further features in the described preferred embodiments the decarboxylase is a glutamate decarboxylase.

According to still further features in the described preferred embodiments the ligase is a glutamate-ethylamine ligase.

According to still further features in the described preferred embodiments the transferase is selected from the group consisting of glutamate n-acetyltransferase and adenylyltransferase.

According to still further features in the described preferred embodiments the aminomutase is a glutamate-1-semialdehyde 2,1-aminomutase.

According to still further features in the described preferred embodiments wherein the agent is at least one co-factor of a glutamate modifying enzyme.

According to still further features in the described preferred embodiments the co-factor is selected from the group consisting of oxaloacetate, pyruvate, NAD⁺, NADP⁺, 2-oxohexanedioic acid, 2-oxo-3-sulfopropionate, 2-oxo-3-sulfinopropionic acid, 2-oxo-3-phenylpropionic acid, 3-indole-2-oxopropionic acid, 3-(4-hydroxyphenyl)-2-oxopropionic acid, 4-methylsulfonyl-2-oxobutyric acid, 3-hydroxy-2-oxopropionic acid, 5-oxopentanoate, 6-oxo-hexanoate, glyoxalate, 4-oxobutanoate, α-ketoisocaproate, α-ketoisovalerate, α-keto-β-methylvalerate, succinic semialdehyde-(-4-oxobutyrate), pyridoxal phosphate, pyridoxal phosphate precursor and 3-oxoisobutanoate.

According to still further features in the described preferred embodiments the agent is a modified glutamate converting enzyme being selected incapable of converting the modified glutamate into glutamate and/or a modification thereof.

According to still further features in the described preferred embodiments the modified glutamate converting enzyme is an α-ketoglutarate dehydrogenase.

According to still further features in the described preferred embodiments the agent is a co-factor of a modified glutamate converting enzyme being selected incapable of converting the modified glutamate into glutamate.

According to still further features in the described preferred embodiments the agent is selected from the group consisting of lipoic acid, lipoic acid precursor, thiamine pyrophosphate, thiamine pyrophosphate, pyridoxal phosphate and pyridoxal phosphate precursor.

According to still further features in the described preferred embodiments the agent includes a glutamate modifying enzyme and a co-factor thereof.

According to still further features in the described preferred embodiments the agent includes a glutamate modifying enzyme and a modified glutamate converting enzyme being selected incapable of converting the modified glutamate into glutamate.

According to still further features in the described preferred embodiments the agent includes a co-factor of a glutamate modifying enzyme and a modified glutamate converting enzyme being selected incapable of converting the modified glutamate into glutamate.

According to still further features in the described preferred embodiments the agent includes a co-factor of a glutamate modifying enzyme, a modified glutamate converting enzyme being selected incapable of converting the modified glutamate into glutamate and a co-factor thereof.

According to still further features in the described preferred embodiments the agent includes a glutamate modifying enzyme, a co-factor thereof, a modified glutamate converting enzyme being selected incapable of converting the modified glutamate into glutamate and a co-factor thereof.

According to still further features in the described preferred embodiments the agent includes a glutamate modifying enzyme, a co-factor thereof, and a modified glutamate converting enzyme being selected incapable of converting the modified glutamate into glutamate.

According to still further features in the described preferred embodiments the agent includes a glutamate modifying enzyme, a modified glutamate converting enzyme being selected incapable of converting the modified glutamate into glutamate and a co-factor thereof.

According to still further features in the described preferred embodiments the agent includes a glutamate modifying enzyme and a co-factor of a modified glutamate converting enzyme being selected incapable of converting the modified glutamate into glutamate.

According to still further features in the described preferred embodiments the agent includes a modified glutamate converting enzyme being selected incapable of converting the modified glutamate into glutamate and a co-factor thereof.

According to still further features in the described preferred embodiments the agent includes a co-factor of a glutamate modifying enzyme and a co-factor of a modified glutamate converting enzyme being selected incapable of converting the modified glutamate into glutamate.

According to still further features in the described preferred embodiments the administering is effected at a concentration of the agent not exceeding 1 g/Kg body weight/hour.

According to still further features in the described preferred embodiments the obtaining the blood sample is effected from:

a matching blood type donor;
a nonmatching blood type donor; and/or
the subject in need thereof.

According to still further features in the described preferred embodiments wherein the agent is at least one inhibitor of a glutamate synthesizing enzyme.

According to still further features in the described preferred embodiments the inhibitor is selected from the group consisting of gamma-Acetylenic GABA, GABAculine, L-canaline, 2-amino-4-(aminooxy)-n-butanoic acid, 3-Chloro-4-aminobutanoate, 3-Phenyl-4-aminobutanoate, Isonicotinic hydrazide; (S)-3-Amino-2-methylpropanoate, Phenylhydrazine; 4-Fluorophenyl)alanine, Adipate, Azaleic acid, Caproate, 3-Methylglutarate, Dimethylglutarate, Diethylglutarate, Pimelate, 2-Oxoglutamate, 3-Methyl-2-benzothiazolone hydrazone hydrochloride, Phenylpyruvate, 4-hydroxyphanylpyruvate, Prephenate and Indole pyruvate.

The present invention successfully addresses the shortcomings of the presently known configurations by providing methods and compositions for protecting neuronal tissue from damage induced by elevated glutamate levels

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the Drawings:

FIGS. 1A-C are graphs showing a dual probe microdialysis of Glu in rat brain striatum. FIG. 1A shows a time course of Glu diffusion to the recovery probe. Glu (1M) was continuously perfused at a rate of 2 μl/min from the delivery probe while perfusing artificial CSF at the same rate of 2 μl/min through the recovery probe for the entire duration of the experiments. The results of 4 experiments were normalized to the maximal value and presented as mean±standard deviation. The broken line shows the expected steady state. FIG. 1B shows the impact of intravenous Glu on brain Glu diffusion to the recovery probe. Microdialysis of 1M Glu along with an intravenous Glu injection performed at t=100 min for 30 min (30 μmoles/min/100 g). The left ordinate shows the concentrations of Glu in the recovery probe while the right ordinate shows the blood Glu concentrations. FIG. 1c shows the effect of oxaloacetate, a blood Glu scavenger, on brain Glu. Dual-probe microdialysis of 1M Glutamate along with an intravenous OxAc injection at t=110 min for a duration of 30 min at a rate of 30 μl/min/100 g and 30 μmoles/min/100 g. The left ordinate shows the concentration of Glu in the recovery probe (diamonds) while the right ordinate shows the blood Glu concentration (rectangles). The results of 2 experiments were normalized to the maximal value at 80 min and presented as mean±standard deviation;

FIGS. 2A-B are line graphs showing the effects of stress hormones and stress on blood glutamate and glucose levels. A: Cortisol (25 mg/kg) was injected intraperitoneally (triangles). Noradrenaline was infused intravenously for 30 min at 2 μg/100 g/min and 1 ml/100 g (rectangles). Adrenaline was infused intravenously for 30 min at 10 g/kg/min and 1 ml/100 g (diamonds). The data presented are normalized to the respective basal blood values and shown as mean±standard deviation (n=3). B: Effects of an intracranial insertion of a microdialysis probe (without any fluid perfusion) on blood Glu (diamonds) and glucose (rectangles) levels. The data presented are normalized to the respective basal blood values and shown as mean±standard deviation (n=4);

FIGS. 3A-C are graphs showing the effects of stress on the spontaneous recovery from traumatic brain injury. FIG. 3A illustrates TBI protocol and times of neurological severity score (NSS) assessment and removal of blood samples (asterisks) FIG. 4B is a histogram showing the effects of propranolol (10 mg/kg i.p) on the spontaneous recovery of rats submitted to TBI. Propranolol was injected 60 min before the infliction of TBI. The neurological severity scores were measured at 1 and 24 hours following TBI. The scores shown on the ordinate correspond to NSS averages+/−standard error of the mean. Control (n=33); propranolol (n=8). Bonferroni's multiple comparison tests: saline 1 h vs saline 24 h p<0.001; saline 1 h vs propranolol pretreatment p>0.05; saline 1 h vs propranolol post-TBI p<0.001. FIG. 3C is a graph showing the effects of propranolol on the blood Glu (rectangles) and glucose (triangles) levels of rats submitted to TBI (n=8). Propranolol was injected at a dose of 10 mg/kg i.p. 60 min before the infliction of TBI. Glu and glucose levels were measured at 1, 60, 120 and 150 min following TBI. Glu levels were normalized to the value at t=1 min.;

FIGS. 4A-C are graphs depicting the effects of a blood Glu scavenger and of Glu on the recovery from TBI. Rats were injected intravenously for a total duration of 30 min (as in FIG. 3A) either with saline (30 μl/min/100 g), OxAc (30 μmoles/min/100 g) or OxAc+Glu (30 μmoles/min/100 g each). Twenty four and forty eight hours later, rats were submitted to various tests to establish the NSS. The scores shown above each column correspond to NSS averages with bars indicating the standard error of the mean. FIG. 4A—The groups from left to right are as follows: control saline (n=33) at 1 hour and 24 h; OxAc (n=32); Glu (n=32); OxAc+Glu (n=17) at 24 h and 48 h; One way ANOVA and Bonferroni multiple comparison tests: At 24 h: saline versus OxAc: p<0.05; saline versus Glu: p<0.01; Glu versus OxAc: p<0.001; saline versus OxAc+Glu: p>0.05. At 48 h: saline versus OxAc: p<0.001; OxAc versus Glu: p<0.001; OxAc versus OxAc+Glu: p<0.05; FIG. 4B—Blood Glu levels following a traumatic brain injury. Glu levels are expressed as the percent of the basal Glu levels measured before TBI. Blood aliquots are removed 60 min after TBI, at 75 min (i.e 15 min of intravenous injection of either saline 30 μl/min/100 g or 30 μmoles/min/100 g OxAc) and at 90 min (end of intravenous treatment). The results are presented as mean±standard error of the mean (n=12-13). Paired t test: 0 versus 60 min saline p=5.10⁻⁴; 0 versus 60 min OxAc p=0.10⁻⁴; 60 min versus 75 min saline: p=0.59; 60 min versus 75 min OxAc: p=3.10⁻³; 75 min versus 90 min saline: p=0.89; 75 min versus 90 min OxAc: p=2.10⁻⁴; C: Time course of the recovery from TBI in saline (n=7) and OxAc-treated (n=7) rats as monitored by NSS values presented here as mean±standard deviation. Student t test for saline versus OxAc at 25 days: p=0.006;

FIG. 5 is a graph showing correlation between the decrease of blood Glu levels and the improvement of NSS. The percent blood Glu decrease and the percent NSS decrease of individual rats were calculated respectively as follows:

(Gluₜ₌₀−Glu_{t=90 min})/Gluₜ₌₀, (NSS_{t=1 h}−NSS_{t=24 h})/NSS_{t=1 h};

FIG. 6 is a graph depicting brain edema formation at 120 min and 24 h following TBI and treatment with either saline 30 μl/min/100 g or 30 μmoles/min/100 g OxAc, as determined by assessing water content. The columns represent mean±standard deviation; 120 min: saline (n=6); OxAc (n=6); p=0.09; 24 h: saline (n=7); OxAc (n=6) p=0.008; The p values correspond to unpaired t tests;

FIGS. 7A-B are graphs showing the effect of a beta 1 antagonist, metaprolol on blood Glu levels and NSS. Metaprolol (15 mg/kg) was injected into 4 naïve rats and the levels of blood Glu were determined at time=0 and every subsequent 30 min until 120 min. Rats (n=5) were preinjected with 15 mg/kg metaprolol and submitted to TBI. The rats NSS were measured after 24 and 48 h The results are presented as mean±standard deviation; and

FIG. 8 is a graph showing the effect of an alpha 1 agonist, phenylephrine on blood Glu levels. Phenylephrine was infused intravenously for 30 min into rats (n=4) at a rate 0.25 mg/0.5 ml/100 g/30 min and the levels of blood Glu were determined at time=0 and every subsequent 30 min until 120 min. The results are presented as mean±standard deviation.

### DESCRIPTION OF THE PREFERRED EMBODIMENTS

The present invention is of compositions and methods using same for reducing the levels of extracellular glutamate in the brain of a subject. Specifically, the present invention can be used to treat acute and chronic brain diseases in which elevated levels of glutamate are detrimental to the subject, such as in ischemic conditions.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Abnormally high glutamate levels in brain interstitial and cerebrospinal fluids are the hallmark of several neurodegenerative conditions. Numerous approaches to reduce glutamate excitotoxicity include development of glutamate receptor antagonists and up-regulation of glutamate transporters. While the first are limited by adverse and even lethal effects probably due to poor selectivity, none of the latter have been successful in providing a viable therapeutic approach for lowering glutamate levels.

The present inventor has previously designed a novel therapeutic modality to clinical conditions associated with elevated extracellular brain glutamate levels which is effected by increasing brain to blood glutamate efflux (see PCT IL 03/00634).

While reducing the present invention to practice the present inventor uncovered that stress conditions may facilitate this brain to blood glutamate efflux, suggesting the use of stress hormones for reducing brain glutamate levels.

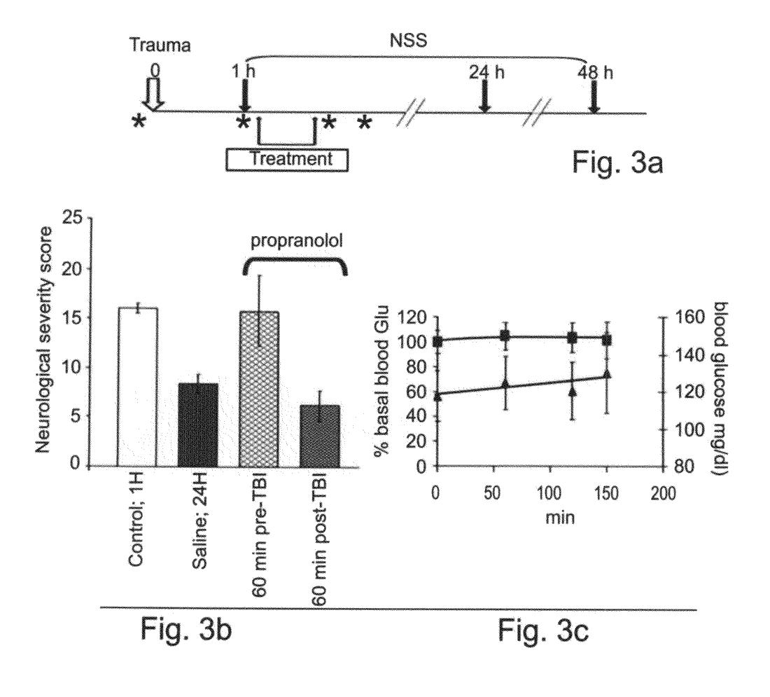

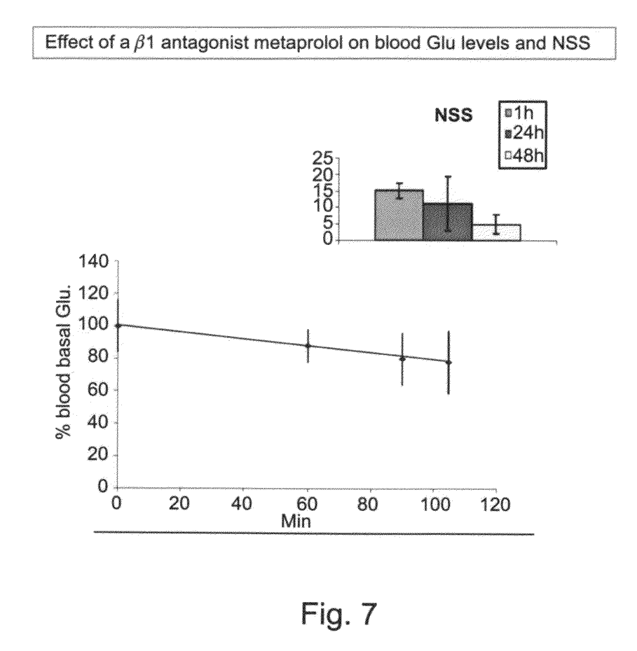

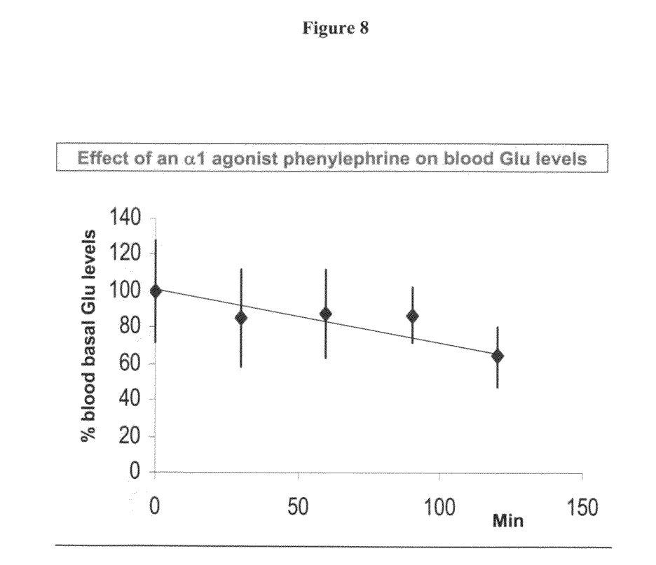

As is illustrated hereinbelow and in the Examples section which follows, stress-mediated efflux accounts for the spontaneous though partial recovery of rats submitted to traumatic brain injury since recovery since it is prevented by increasing blood glutamate or propranolol administration (a highly potent selective beta-adrenergic receptor antagonist), but is improved by the intravenous administration of oxaloacetate, a blood glutamate scavenger. Additionally administration of phenylephrine (an alpha-1 adrenergic receptor agonist injected intravenously at 0.1 mg/100 gr rat body-weight/30 min, FIG. 8) reduced blood basal glutamate levels by 40% while the adrenergic antagonist propranolol had practically no effect on blood glutamate levels (FIGS. 3A-C and 4A-C). Moreover, metaprolol, a beta 1 adrenergic receptor antagonist reduced in vivo rat blood glutamate by 40% (FIGS. 7A-B). These results indicate that different stress pathways have different effects (and even contrasting effects e.g., beta 1 and beta 2) on blood glutamate levels and thus appropriate agonists and antagonists of same can be used for modulating blood glutamate levels and as such used for the treatment of clinical conditions characterized by elevated extracellular brain glutamate levels.

Thus, according to one aspect of the present invention, there is provided a method of reducing extracellular brain glutamate levels.

The method is effected by administering to a subject in need thereof an agent capable of modulating stress hormone activity thereby reducing blood glutamate levels, thereby reducing extracellular brain glutamate levels.

Preferred individual subjects according to the present invention are mammals, preferably human subjects.

As used herein the phrase “a subject in need thereof” refers to a subject who is exposed or may be exposed to the effects of abnormally high brain glutamate levels.

As used herein “high brain glutamate levels” refer to a concentration above the resting value of 1 μM (e.g., 5-100 μM).

Methods of determining brain glutamate levels are well known in the art and further described hereinbelow and in the examples section which follows.

Agents capable of modulating stress hormone activity, may be agents capable of upregulating stress hormone activity (agonists) thereby reducing blood glutamate levels.

Alternatively, agents capable of modulating stress hormone activity, may be agents capable of downregulating stress hormone activity (e.g., antagonists) thereby reducing blood glutamate levels.

For example, an agent capable of modulating an activity of a stress hormone is a stress hormone agonist (synthetic or natural, e.g., alpha 1 or alpha 2 or beta 1 adrenergic receptor agonist) or a downstream effector thereof. Alternatively, the agent may be a natural or synthetic stress hormone antagonist (e.g., beta 1 adrenergic receptor antagonist).

As used herein the phrase “stress hormone” relates to a hormone which is secreted following stress. Stress involves the activation of two systems: the sympathetic adrenomedullary system with the secretion of epinephrine and norepinephrine, and the hypothalamic pituitary adrenocortical (HPA) system with the secretion of cortisol. In the latter system, the stress hormones and effector systems include among others the corticotropin releasing hormone (CRH), the primary secretagogue for ACTH, and arginine vasopressin (AVP) that modulate ACTH release. The binding of ACTH to its receptors in the adrenal cortex causes the release of glucocorticoids such as cortisol and corticosterone.

Examples of stress hormones which may be modulated in accordance with the present invention are listed in Table 1 below.

TABLE 1

| | | | Natural/ | |
|---|---|---|---|---|
| Stress Hormone | Definition | Receptor Agonist | on natural | Reference |
| Epinephrine (adrenaline) | adrenocortical stress | beta ½ adrenergic | natural | Sigola L B, and Zinyama |
| | hormone | | | R B, Immunology. |
| | (Catecholamine) | | | 2000 July; 100(3): 359-63) |
| 3,4-dihydroxyphenylglycol | neuronal deaminated | metabotropic glutamate | natural | Rouach Nand Nicoll |
| (DHPG) | metabolite of | 1 (mGlu1)/mGlu 5 | | R A. Eur J Neurosci. |
| | norepinephrine | | | 2003 August; 18(4): 1017-20 |
| isoprenaline | synthetic derivative of | beta1/beta2 adrenergic | synthetic | Sigola L B, and Zinyama R B, |
| | noradrenaline | | | Immunology. 2000 |
| | | | | July; 100(3): 359-63). |
| salbutamol (Ventolin) | | selective beta2 | synthetic | Sigola L B, and Zinyama R B, |
| | | adrenergic | | Immunology. 2000 |
| | | | | July; 100(3): 359-63). |
| dobutamine | | selective beta1 | synthetic | Sigola L B, and Zinyama R B, |
| | | adrenergic | | Immunology. 2000 |
| | | | | July; 100(3): 359-63). |
| phenylephrine | | alpha 1 adrenergic | synthetic | Meck, J. V. et al., Am |
| | | | | J Physiol Heart Circ |
| | | | | Physiol 286: H1486- |
| | | | | H1495, 2004.) |
| cortisone | Glucocorticoid | receptor-inactive | natural | John W. Funder |
| | hormone (steroid). A | | | Aust Prescr |
| | metabolite of cortisol | | | 1996; 19: 41-4 |
| Aldosterone | mineralocorticoid | Mineralocorticoid | natural | John W. Funder Aust |
| | steroid hormones | receptor > | | Prescr 1996; 19: 41-4 |
| | | glucocorticoid receptor | | |
| RU28362 | Synthetic | glucocorticoid receptor | Synthetic | Ginsberg A B, et al., J |
| | glucocorticoid | (GR) | | Neuroendocrinol. |
| | | | | 2006 February; 18(2): 129-38. |
| dexamethasone (DEX), | Synthetic | glucocorticoid receptor | synthetic | Komatsuzaki Y, et |
| | glucocorticoid | (GR) | | al., Biochem Biophys |
| | | | | Res Commun. 2005 |
| | | | | Oct. 7; 335(4): 1002-7 |
| beta-endorphin | opioid neuropeptide | Mu1-opioid receptor > | natural | Armstrong D W 3rd |
| | | Mu2 and Delta-opioid | | and Hatfield B D. |
| | | receptors > Kappa1- | | Eur J Appl Physiol. |
| | | opioid receptors. | | 2006 Feb. 9; : 1-9 |
| Angiotensin II (AII) | Vasoconstrictor | Angiotensin II receptor | natural | Wan Y., et al., Sheng |
| | octapeptide | | | Li Xue Bao. 1996 |
| | stimulator of | | | December; 48(6): 521-8 |
| | aldosterone secretion | | | |
| Prolactin (PRL) | pituitary gonadotropic | prolactin receptor | natural | Marquez C, et al., |
| | hormone | | | Behav Brain Res. |
| | | | | 2006 Mar. 15; 168(1): 13-22 |
| adrenocorticotropic hormone | Pituitary | ACTH receptor | natural | Muzii L, J Am |
| ACTH (corticotrophin) | hormone | | | Assoc Gynecol Laparosc. 1996 |
| | | | | February; 3(2): 229-34 |
| testosterone | Androgenic steroid | androgen receptor | natural | Gomez F, |
| | | | | Endocrinology. 2004 |
| | | | | January; 145(1): 59-70 |
| Growth hormone (GH) | Somatotropic pituitary | growth hormone | | Wagner K, et al., Int |
| | hormone | receptor (GHR) | | J Cancer. 2005 Dec. 27 |
| somatrem | synthetic growth | growth hormone | synthetic | Bayol, A, et al., |
| | hormone | receptor (GHR) | | Pharmeuropa Bio. |
| | | | | 2004 December; 2004(1): 35-45. |

Methods of selecting preferred stress hormones which are capable of reducing blood glutamate levels are further described hereinbelow and in the examples section which follows. Generally, these methods may involve biochemical, immunological and spectrochemical assays which are well known in the art and exemplified in the Examples section which follows (see Experimental Procedures).

As shown in 4A-C administration of oxaloacetate (OxAc) caused a larger (synergistic) decrease of blood Glu levels than that caused by stress (following traumatic brain injury).

Thus according to a preferred embodiment of this aspect of the present invention the method further comprising administering another agent capable of reducing blood glutamate levels prior to, concomitant with of following administration of the stress hormone.

Preferably, co-administration is expected to have a synergistic effect on blood glutamate reduction. Preferred level of reducing blood glutamate is 50%.

An agent, which is capable of reducing blood glutamate according to this aspect of the present invention includes any glutamate modifying enzyme and/or a co-factor thereof.

As used herein “a glutamate modifying enzyme” is an enzyme, which utilizes glutamate as a substrate and produces a glutamate reaction product. A glutamate modifying enzyme can be a natural occurring enzyme or an enzyme which has been modified to obtain improved features, such as higher affinity to glutamate than to a modified glutamate, stability under physiological conditions, solubility, enhanced enantioselectivity, increased thermostability and the like as is further described hereinunder.

Numerous glutamate modifying enzymes are known in the art. For example, transaminases, which play a central role in amino acid metabolism and generally funnel α-amino groups from a variety of amino acids to α-ketoglutarate.

Examples of transaminases include but are not limited to glutamate oxaloacetate transaminases, glutamate pyruvate transaminases, acetylornithine transaminases, ornithine-oxo-acid transaminases, succinyldiaminopimelate transaminases, 4-aminobutyrate transaminases, alanine transaminases, (s)-3-amino-2-methylpropionate transaminases, 4-hydroxyglutamate transaminases, diiodotyrosine transaminases, thyroid-hormone transaminases, tryptophan transaminases, diamine transaminases, cysteine transaminases, L-Lysine 6-transaminases, histidine transaminases, 2-aminoadipate transaminases, glycine transaminases, branched-chain-amino-acid transaminases, 5-aminovalerate transaminases, dihydroxyphenylalanine transaminases, tyrosine transaminases, phosphoserine transaminases, taurine transaminases, aromatic-amino-acid transaminases, aromatic-amino-acid-glyoxylate transaminases, leucine transaminases, 2-aminohexanoate transaminases, ornithine (lysine) transaminases, kynurenine-oxoglutarate transaminases, D-4-hydroxyphenylglycine transaminases, cysteine-conjugate transaminases, 2,5-diaminovalerate transaminases, histidinol-phosphate transaminases, diaminobutyrate-2-oxoglutarate transaminases, UDP-2-acetamido-4-amino-2,4,6-trideoxyglucose transaminases and aspartate transaminases.

Other examples of glutamate modifying enzymes include but are not limited to glutamate dehydrogenases, which generate ammonium ion from glutamate by oxidative deamination; decarboxylases such as glutamate decarboxylase; ligases such as glutamate-ethylamine ligase, glutamate-cysteine ligase; transferases such as glutamate N-acetyltransferase and N2-acetyl-L-ornithine, adenylyltransferase; aminomutases such as glutamate-1-semialdehyde 2,1-aminomutase and glutamate racemase [Glavas and Tanner (2001) Biochemistry 40(21):6199-204)].

It will be appreciated that artificially modified enzymes can also be used according to this aspect of the present invention.

Modification of enzymes can be effected using numerous protein directed evolution technologies known in the art [for review see Kuchner and Arnold (1997) TIBTECH 15:523-530].

Typically, directed enzyme evolution begins with the creation of a library of mutated genes. Gene products that show improvement with respect to the desired property or set of properties are identified by selection or screening, and the gene(s) encoding those enzymes are subjected to further cycles of mutation and screening in order to accumulate beneficial mutations. This evolution can involve few or many generations, depending on the progress observed in each generation.

Preferably, for successful directed evolution a number of requirements are met; the functional expression of the enzyme in a suitable microbial host; the availability of a screen (or selection) sensitive to the desired properties; and the identification of a workable evolution strategy.

Examples of mutagenesis methods which can be used in enzyme directed evolution according to this aspect of the present invention include but are not limited to UV irradiation, chemical mutagenesis, poisoned nucleotides, mutator strains [Liao (1986) Proc. Natl. Acad. Sci. U.S.A 83:576-80], error prone PCR [Chen (1993) Proc. Natl. Acad. Sci. U.S.A 90:5618-5622], DNA shuffling [Stemmer (1994) Nature 370:389-91], cassette [Strausberg (1995) Biotechnology 13:669-73], and a combination thereof [Moore (1996) Nat. Biotechnol. 14:458-467; Moore (1997) J. Mol. Biol. 272:336-347].

Screening and selection methods are well known in the art [for review see Zhao and Arnold (1997) Curr. Opin. Struct. Biol. 7:480-485; Hilvert and Kast (1997) Curr. Opin. Struct. Biol. 7:470-479]. Typically, selections are attractive for searching larger libraries of variants, but are difficult to device for enzymes that are not critical to the survival of the host organism. Further more, organisms are may evade imposed selective pressure by unexpected mechanisms. Less stringent functional complementation can be useful in identifying variants which retain biological activity in libraries generated using relatively high mutagenic rates [Suzuki (1996) Mol. Diversity. 2:111-118; Shafikhani (1997) Biol. Techniques 23:304-310; Zhao and Arnold (1997) Curr. Opin. Struct. Biol. 7:480-485].

As described hereinabove, the agent according to this aspect of the present invention, can include one or more co-factors of glutamate modifying enzymes, which can accelerate activity of the latter (Vₘₐₓ). These can be administered in order to enhance activity of endogenous glutamate modifying enzymes or in conjunction with glutamate modifying enzymes (described hereinabove).

Co-factors of glutamate-modifying enzymes include but are not limited to oxaloacetate, pyruvate, NAD⁺, NADP⁺, 2-oxohexanedioic acid, 2-oxo-3-sulfopropionate, 2-oxo-3-sulfinopropionic acid, 2-oxo-3-phenylpropionic acid, 3-indole-2-oxopropionic acid, 3-(4-hydroxyphenyl)-2-oxopropionic acid, 4-methylsulfonyl-2-oxobutyric acid, 3-hydroxy-2-oxopropionic acid, 5-oxopentanoate, 6-oxo-hexanoate, glyoxalate, 4-oxobutanoate, α-ketoisocaproate, α-ketoisovalerate, α-keto-β-methylvalerate, succinic semialdehyde-(-4-oxobutyrate), 3-oxoisobutanoate, pyridoxal phosphate, 5-oxopentanoate, 6-oxohexanoate and their artificially modified derivatives (e.g., esters).

Since modified glutamate (i.e., glutamate reaction product) can be reversibly modified (i.e., interconverted) to glutamate, the agent, according to this aspect of the present invention, preferably includes a modified glutamate converting enzyme which is incapable of converting the modified glutamate into glutamate to thereby insuring continual metabolism of glutamate.

Examples of modified glutamate converting enzymes include but are not limited to α-ketoglutarate dehydrogenase, and the like.

Modified glutamate converting enzymes can also include glutamate modifying enzymes artificially modified to possess lower affinity for glutamate reaction product than for glutamate. For example, the E. coli GOT (GenBank Accession No. D90731.1) is characterized by an affinity for glutamate of about 8 mM and an affinity for 2-ketoglutarate of about 0.2 mM. A human enzyme or a humanized enzyme characterized by such affinities is preferably used according to this aspect of the present invention such as described by Doyle et al. in Biochem J. 1990 270(3):651-7.

Optionally, co-factors of modified glutamate converting enzymes can be included in the agent according to this aspect of the present invention. Examples of co-factors of modified glutamate converting enzymes include but are not limited to lipoic acid and its precursors, thiamine pyrophosphate and its precursors, pyridoxal phosphate and its precursors and the like.

It will be appreciated that the agent according to this aspect of the present invention may also include inhibitors of glutamate synthesizing enzymes (e.g., phosphate activated glutaminase). Numerous inhibitors of glutamate producing enzymes are known in the art. Examples include but are not limited gabapentin which has been shown to modulate the activity of branched chain aminotransferases [Taylor (1997) Rev. Neurol. 153(1):S39-45] and aspirin at high doses (i.e., 4-6 g/day) a neuroprotective drug against glutamate excitotoxicity [Gomes (1998) Med. J. India 11:14-17]. Other inhibitors may be identified in the publicly available BRENDA, a comprehensive enzyme information system [www.brenda.uni-koeln.de/]. Examples include but are not limited to, gamma-Acetylenic GABA, GABAculine, L-canaline, 2-amino-4-(aminooxy)-n-butanoic acid; 3-Chloro-4-aminobutanoate; 3-Phenyl-4-aminobutanoate; Isonicotinic hydrazide; (S)-3-Amino-2-methylpropanoate; Phenylhydrazine; 4-Fluorophenyl)alanine; Adipate, Azelaic acid, Caproate, 3-Methylglutarate, Dimethylglutarate, Diethylglutarate, Pimelate, 2-Oxoglutamate; 3-Methyl-2-benzothiazolone hydrazone hydrochloride; Phenylpyruvate, 4-Hydroxyphenylpyruvate, Prephenate, Indole pyruvate).

Although each of the components described hereinabove may comprise the agent of the present invention, it will be appreciated that for optimal blood-glutamate reducing activity, the agent may include a combination of the above described components (i.e., glutamate modified enzyme, co-factor thereof, modified glutamate converting enzyme and co-factor thereof).

For optimal brain-to-blood glutamate efflux the agent is preferably selected capable of reducing plasma glutamate levels rather than blood cell glutamate levels.

Thus, according to preferred embodiments of this aspect of the present invention the agent includes oxaloacetate and pyruvate. Preferably, the agent is administered at a concentration not exceeding 1 g/kg×hour.

According to a presently preferred embodiment of this aspect of the present invention the agent includes.

In some cases, the agent administered is modified in order to increase the therapeutic effect or reduce unwanted side effects. For example, administration of oxaloacetate diethylester is favorable over administration of oxaloacetate alone since oxaloacetate exerts its therapeutic potential at relatively high concentrations and requires full titration of its carboxyl moieties with sodium hydroxide at 2:1 stoichiometric ratio which presents unacceptable electrolyte load above safe levels.

The agents of the present invention (i.e., stress hormone and optionally other agents described hereinabove such as oxaloacetate) can be administered to a subject using any one of several suitable administration modes which are further described hereinbelow with respect to the pharmaceutical compositions of the present invention.

The agent utilized by the method of the present invention can be administered to an individual subject per se, or as part of a pharmaceutical composition where it is mixed with a pharmaceutically acceptable carrier.

As used herein a “pharmaceutical composition” refers to a preparation of one or more of the active ingredients described hereinabove along with other components such as physiologically suitable carriers and excipients, penetrants etc. The purpose of a pharmaceutical composition is to facilitate administration of a compound to an organism.

Herein the term “active ingredient” refers to the preparation accountable for the biological effect (e.g., the glutamate modifying enzyme, and/or cofactors thereof).

Hereinafter, the phrases “physiologically acceptable carrier” and “pharmaceutically acceptable carrier” are interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration of the pharmaceutical composition of the present invention may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any pharmaceutical composition used by the treatment method of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

The Examples section, which follows provides further guidance as to suitable dosages.

Depending on the severity and responsiveness of the condition to be treated, dosing can be of a single or a plurality of administrations, with course of treatment lasting from several days to several weeks or until cure is effected or diminution of the disease state or symptoms is achieved.

The amount of the pharmaceutical composition to be administered will, of course, be dependent on the subject being treated, the severity of the affliction, the manner of administration, the judgment of the prescribing physician, etc.

For example, dosing can be determined by measuring brain pressure, which is known to be affected by extracellular brain glutamate (Poon W S, Ng S C, Chan M T, Leung C H, Lam J M. Neurochemical changes in ventilated head-injured patients with cerebral perfusion pressure treatment failure. Acta Neurochir Suppl. 2002; 81:335-8. Chan T V, Ng S C, Lam J M, Poon W S, Gin T. Monitoring of autoregulation using intracerebral microdialysis in patients with severe head injury. Acta Neurochir Suppl. 2005; 95:113-6); or by microdialysis. Alternatively, glutamate levels may be determined in the CSF using invasive as well as non-invasive means (e.g., magnetic resonance spectroscopy (Pan J W, Mason G F, Pohost G M, Hetherington H P. Spectroscopic imaging of human brain glutamate by water-suppressed J-refocused coherence transfer at 4.1T Magn. Reson. Med. 1996, 36, 7-12).

Typically, administration of the agents of the present invention is effected as soon as feasibly possible and repeated as needed according to brain glutamate levels, depending on the type of neurological condition. For example, in traumatic brain injury and stroke, high glutamate levels in brain fluids are a sign of a still ongoing glutamate mediated neuropathological action. This corresponds to an ongoing secondary elevation of glutamate or a malignant stroke. It has been shown that there is a tight correlation between the prolonged (hours-days) and high glutamate levels in brain and neurological deterioration. Therefore, the present invention envisages administration of the agents either upon appearance of first symptoms of the neurological condition or repetitively at later stages.

Typical concentration of adrenaline is 0.2 to 0.5 mg; isoproterenol 200 μg/ml; metaprolol 5 mg/ml, phenylephrine 3 mg/ml (Goodman and Gilman The pharmacological basis of therapeutics).

Compositions including the preparation of the present invention formulated in a compatible pharmaceutical carrier may also be prepared, placed in an appropriate container, and labeled for treatment of an indicated condition.

For example, the stress hormone may be placed in a single container and at least one of the above-described agents (oxaloacetate) may be placed in another container. Alternatively the two may be placed in a single container.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

The agents of the present invention can be utilized in treating (i.e., reducing or preventing or substantially decreasing elevated concentrations of extracellular brain glutamate) of a variety of clinical conditions associated with elevated levels of extracellular brain glutamate such as brain anoxia, stroke, perinatal brain damage, traumatic brain injury, bacterial meningitis, subarachnoid hemorrhage, epilepsy, acute liver failure, glaucoma, amyotrophic lateral sclerosis, HIV, dementia, amyotrophic lateral sclerosis (ALS), spastic conditions, open heart surgery, aneurism surgery, coronary artery bypass grafting and Alzheimer's disease.

It will be appreciate that fast acting pharmaceutical compositions and administration routes described hereinabove are preferably used in treating brain anoxia, stroke, perinatal brain damage, traumatic head injury, bacterial meningitis, subarachnoid hemorrhage, epilepsy, acute liver failure, open heart surgery, aneurysm surgery, coronary artery bypass grafting. When continuous administration is required a continuous drug release is preferred provided that endogenous production in the depleted organ does not occur.

Blood cells which are isolated from the body may be depleted of glutamate and returned to the body to thereby induce a decrease in extracellular brain glutamate levels.

Thus according to another aspect of the present invention there is provided an additional method of reducing extracellular bran glutamate levels.

The method according to this aspect of the present invention is based upon the rational that glutamate depleted blood cells are capable of rapidly pumping plasma glutamate towards the original cell/plasma glutamate concentration ratio (i.e., substantially 4:1) upon transfusion into a host subject, thereby promoting brain-to-blood (i.e., brain-to-plasma) glutamate efflux and reducing extracellular brain glutamate concentration.

The method according to this aspect of the present invention is effected by treating blood samples derived from a subject with glutamate reducing agents such as those described hereinabove, isolating cells from the blood sample and returning the cells to the subject.

Preferably the blood sample according to this aspect of the present invention is obtained from the subject for further autologous transfusion. This reduces the risk of infectious diseases such as hepatitis, which can be transferred by blood transfusions.

It will be appreciated that matching blood type (i.e., matching blood group) samples from syngeneic donors can be used for homologous transfusion although non-matching blood type samples from allogeneic donors may also be used in conjunction with a deantigenation procedure. A number of methods of deantigenation of blood group epitopes on erythrocytes (i.e., seroconversion) are known in the art such as disclosed in U.S. Pat. Nos. 5,731,426 and 5,633,130.

Blood samples are contacted with the stress hormone (and optionally another agent for reducing blood glutamate levels, as described above) of the present invention under conditions suitable for reducing blood glutamate levels to thereby obtain glutamate depleted blood cells (as described herein above and further in Examples 14-15 of the Examples section which follows).

Glutamate depleted blood cells are then separated from plasma by well known separation methods known in the art, such as by centrifugation (see Example 14 of the Examples section).

Once glutamate depleted cells are obtained they are suspended to preferably reach the original blood volume (i.e., concentration).

Suspension of glutamate depleted cells is effected using a blood substitute. “A blood substitute” refers to a blood volume expander which includes an aqueous solution of electrolytes at physiological concentration, a macromolecular oncotic agent, a biological buffer having a buffering capacity in the range of physiological pH, simple nutritive sugar or sugars, magnesium ion in a concentration sufficient to substitute for the flux of calcium across cell membranes. A blood substitute also includes a cardioplegia agent such as potassium ion in a concentration sufficient to prevent or arrest cardiac fibrillation. Numerous blood substitutes are known in the art. Examples include but are not limited to Hespan® (6% hetastarch 0.9% Sodium chloride Injection [Dupont Pharmaceuticals, Wilmington Del.]), Pentaspan (10% pentastarch in 0.9% Sodium chloride Injection [Dupont Pharmaceuticals, Wilmington Del.]) and Macrodex (6% Dextran 70 in 5% Dextrose Injection or 6% Dextran 70 in 0.9% Sodium chloride Injection [Pharmacia, Inc. Piscataway, N.J.]) and Rheomacrodex (10% Dextran 40 in 5% Dextrose Injection or 10% Dextran 40 in 0.9% Sodium chloride Injection [Pharmacia, Inc. Piscataway, N.J.]). These products are known to the medical community for particular FDA approved indications and are extensively described in the volume entitled Physicians' Desk Reference, published annually by Medical Economics Company Inc.

It will be appreciated that treated blood samples may be stored for future use. In this case, however, a sterile preservative anticoagulant such as citratephosphate-dextrose-adenine (CPDA) anticoagulant is preferably added to the blood substitute solution. Also added are a gram-negative antibiotic and a gram-positive antibiotic. Blood is then stored in sterile containers such as pyrogen-free containers at 4° C.

Finally, glutamate depleted blood cell solution is transfused intravenously or intravascularly as a sterile aqueous solution into the host subject, to thereby reduce extracellular brain glutamate levels.

It will be appreciated that previous studies have emphasized the relative impermeability of erythrocytes to extracellular glutamate [Young (1980) Proc. R. Soc. Lond. B. Biol. Sci. 209:355-75; Pico (1992) Int. J. Biochem. Cell Biol. 27(8):761-5 and Culliford (1995) J. Physiol. 489(Pt3):755-65]. However, these were done in the presence of an unfavorable glutamate concentration gradient.

Thus, not only does the present invention exhibits erythrocytes permeability to glutamate, thereby explaining the still unclear blood pool of intracellular glutamate, but provides with a blood exchange strategy which can be utilized in emergency conditions such as stroke and head trauma, in which a rapid reduction in CSF/ISF glutamate is desired.

The above described methodology can be effected using currently available devices such as incubators and centrifuges (see the Examples section for further detail) or a dedicated device which is designed and configured for obtaining a blood sample from the subject, processing it as described above and returning glutamate depleted blood cells to the subject or to a different individual which requires treatment.

Such a device preferably includes a blood inlet, a blood outlet and a chamber for processing blood and retrieving processed blood cells. At least one of the blood inlet and outlet is connected to blood flow tubing, which carries a connector spaced from the device for access to the vascular system of the subject.

Blood treatment devices providing an extracorporeal blood circuit to direct blood to a treatment device from the individual subject, and then to return the blood to the individual subject are well known in the art. Such treatment devices include, but are not limited to hemodialysis units, plasmapheresis units and hemofiltration units, which enable blood flow across a unit, which carries a fixed bed of enzyme or other bioactive agent.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Materials and Experimental Procedures

Animal anesthesia and well being—Experiments were conducted according to the recommendations of the Declarations of Helsinki and Tokyo and to the Guidelines for the Use of Experimental Animals of the European Community. The experiments were approved by the Animal Care Committees of Ben-Gurion University of the Negev and of the Weizmann Institute. Spontaneously breathing male Sprague Dawley rats weighing 200-300 g were anesthetized with a mixture of isoflurane (initial inspired concentration 2%) in 100% oxygen (1l/min). The rectal temperature was maintained at 37° C. using a heating pad and anesthesia was considered as sufficient for surgery when tail reflex was abolished. Catherization of the tail vein was carried out with a BD Neoflon 24 g catheter for allowing fluid infusion. Catherization of the tail artery was performed to allow blood sampling and determination of blood pressure and heart rate. Blood samples were analyzed for pH, pO₂, pCO₂, HCO₃⁻³. Glucose levels were measured with Accu-Chek sensor comfort. After scalp infiltration with bupivacaine 0.5%, it was incised and reflected laterally and a cranial impact of 0.5 J was delivered by a silicone-coated rod which protruded from the center of a free-falling plate as previously described [19]. The impact point was 1-2 mm lateral to the midline on the skull's convexity. Following traumatic brain injury (TBI), the incision was sutured. Following TBI, all rats were laid on their left side in order to recover from anesthesia and to check righting reflex and recovery time as part of the assessment of NSS which was evaluated 1 h after TBI (see below).

Dual-probe microdialysis—Following anesthesia with intraperitoneal urethane (0.125 g/0.2 ml/100 g), rats were implanted with two microdialysis probes inserted in the striatum. Artificial CSF was perfused at a rate of 2 μl/min through the two probes for a duration of 60 min. A 1 M solution of glutamate was then perfused through the delivery probe, at a rate of 2 μl/min for the entire duration of the experiment while perfusing artificial CSF at the same rate of 2 μl/min through a recovery probe, located at 1 mm from the delivery probe. Aliqouots of 40 μl were collected from the recovery probe every 20 min. The recovered Glu was measured using a spectrofluorometric assay (see below).

Glu spectrofluorometric assay—Glu concentration was measured using the fluorometric method of Graham and Aprison [35]. A 20 μl aliquot from microdialysate was added to 480 μl HG buffer containing 15 U of Glu dehydrogenase in 0.2 mM NAD, 0.3M glycine, 0.25 M hydrazine hydrate adjusted to pH 8.6 with 1N H₂SO₄. After incubation for 30-45 min at room temperature, the fluorescence was measured at 460 nm with excitation at 350 nm. A Glu standard curve was established with concentrations ranging from 0-6 μM. All determinations were done at least in duplicates. The results are expressed as mean.+−SD.

Determination of Blood/plasma Glu—Whole blood (200 μl aliquot) were deproteinized by adding an equal volume of ice-cold 1 M perchloric acid (PCA) and then centrifuging at 10000×g for 10 min at 4° C. The pellet was discarded and supernatant collected, adjusted to pH 7.2 with 2M K₂CO₃ and, if needed, stored at −80° C. for later analysis using the above-described assay.

Brain water content—Brain hemispheres were removed 120 min after TBI in some groups while in others, brain tissue samples of approximately 50 mg were excised at 24 h post TBI from a location immediately adjacent to the area of macroscopic damage in the left hemisphere and from a corresponding area in the right hemisphere. These tissue samples were used for determination of water content. Water content was determined from the difference between wet weight (WW) and dry weight (DW). Specifically, after WW of fresh brain tissue samples was obtained, samples were dried in a desiccating oven at 120° C. for 24 h and weighed again to obtain DW. Tissue water content (%) was calculated as (WW−DW)×100/WW.

Neurological severity score—The NSS was determined [19] by a blinded observer. Points are assigned for alterations of motor functions and behavior so that the maximal score of 25 represents severe neurological dysfunction whilst a score of 0 indicates an intact neurological condition. Specifically, the following were assessed: ability to exit from a circle (3 point scale), gait on a wide surface (3 point scale), gait on a narrow surface (4 point scale), effort to remain on a narrow surface (2 point scale), reflexes (5 point scale), seeking behavior (2 point scale), beam walking (3 point scale), and beam balance (3 point scale).

Experimental design—The 1 h assessment of NSS was followed 15 min later by a 30 min-long intravenous administration at a rate of 30 microliter/min/100 g of a solution of either saline, or 1M oxaloacetate, 1M glutamate or their mix. Following treatment, the animals were returned to their cages and given free access to food and water. At 24 h and 48 h, the assessment of NSS was repeated.

Statistical analysis—The a priori hypothesis was that the Glu concentrations in blood samples would differ for treatment groups versus controls. Accordingly, this comparison was made with a t-test (Differences were considered as significant when P<0.05).

The significance of comparisons of the NSS between different groups was assessed using analysis of variance (ANOVA) with Bonferroni post hoc testing. The minimal level of significance accepted was P<0.05.

Data are presented as means±SD or SEM when n>8. Differences were considered as significant when P<0.05.

### Example 1

### Brain Excess Glu Levels are Regulated by a Brain-to-Blood Glu Efflux

Glu efflux from the brain parenchyma interstitial fluid (ISF) to blood was first assessed [10]. The dual-probe brain microdialysis was used and perfused through the delivery probe inserted in the striatum of anesthetized rats a Glu solution while simultaneously perfusing artificial CSF through a recovery probe, located at 1 mm distance. Studies of dual-probe microdialysis describe a tissue delivery of solutes from the delivery probe of 3-6% [12] and a solute recovery of 5% in the recovery probe [13]. As Glu flows out of the first probe, it diffuses through the brain parenchyma where it is taken up into glial cells, neurons and blood capillary endothelial cells. However, as Glu keeps oozing from the delivery probe and saturates the transporters, it eventually reaches the recovery probe if its starting concentration is sufficiently high (>0.5M).

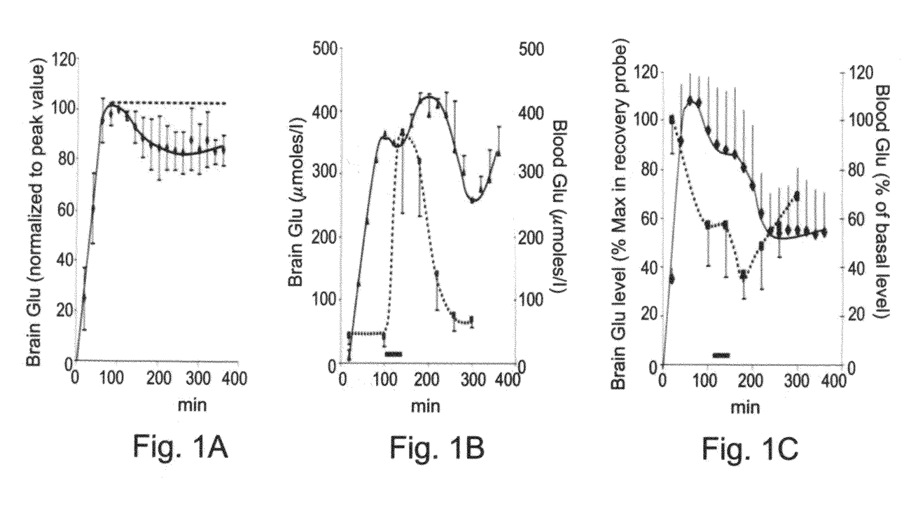

FIG. 1A shows that linearly increasing amounts of Glu arrive with time at the recovery probe. However, following a peak at about 100 min, smaller amounts of Glu reach the recovery probe until a steady state is attained. Interestingly, no such decrease was observed with other solutes such as dopamine or mannitol [12]. At this stage, two interpretations were possible: 1) Glu causes a time and concentration-dependent edematous response of the inter-probe parenchyma that restricts free diffusion in the extracellular space [14] and prevents Glu from reaching the recovery probe. 2) The high Glu concentrations within the inter-probe parenchyma saturate the glial transporters and cause an increased brain-to-blood Glu efflux that reduces the amounts of Glu reaching the recovery probe. One makes here the reasonable assumption that the glial sink for glutamate is significantly smaller than the blood sink.

To confirm the contribution of a brain-to-blood Glu efflux to the control of brain Glu, the microdialysis experiments were combined with an intravenous infusion of a Glu solution performed at 100 min from the onset of the dual-probe microdialysis and for a duration of 30 min.

FIG. 1B shows the results of a typical experiment in which several Glu waves can be monitored via the recovery probe. The first corresponds to a transient Glu peak observed at around 100 min. Following the intravenous administration of Glu, significantly more Glu reaches the recovery probe until a quasi steady state is attained between 180-220 min. One then observes a sharp decline of the amounts of Glu reaching the recovery probe. This decline lasting about 60 min is then followed by a steady increase of the amounts of Glu reaching the recovery probe.

The intuitive interpretation of these data is that the naturally occurring brain-to-blood Glu efflux which is driven by excess brain Glu, is impeded by the high blood Glu concentrations. This leads to the increase of Glu observed between 100 and 220 min, which also rules out the hypothesis of a Glu-driven edematous response. It is only until the high blood Glu concentrations dissipate and establish a renewed brain-to-blood Glu driving force (at a value around 100 □M at ˜220 min), that a brain-to-blood Glu efflux takes place decreasing the amounts of Glu reaching the recovery probe. As the transporters involved in the brain-to-blood Glu efflux become saturated, there is a renewed build-up of ISF Glu that causes more Glu to reach the recovery probe. One could possibly argue that the increased Glu reaching the recovery probe at around 120 min is due to blood Glu entering into the brain through an injured blood brain barrier at the sites of insertion of the microdialysis probes. However, in such an event, one would expect the changes of brain Glu monitored by the recovery probe to faithfully follow the changes of blood Glu. It is clearly not the case.

Next, the blood Glu scavenger OxAc was tested for its ability to cause an increased elimination of excess Glu from the brain inter-probe parenchyma on the background of an already existing brain-to-blood Glu efflux.

FIG. 1C shows that, following a peak of Glu at 80 min and a decrease to a steady state at 80% of the peak Glu value, the administration of OxAc causes a further reduction of the amounts of Glu reaching the recovery probe (by up to 50% of the peak value). These results strengthen the conclusion that intravenous OxAc causes an increased elimination of excess Glu from brain [10]. Surprisingly, the concomitant analysis of blood Glu levels reveals the occurrence of a spontaneous decrease of blood Glu that precedes the administration of OxAc. The increase of blood Glu levels subsequent to the administration of OxAc has been accounted [10] to a compensatory efflux into blood of cytoplasmic Glu from peripheral organs such as liver, brain and muscle which sense the decrease in blood Glu levels.

### Example 2

### Regulation of Blood Glu Levels by Stress

What causes the spontaneous decrease of blood Glu levels? It was suggested that such decrease could be part of a general stress response activating the hypothalamo-pituitary-adrenal (HPA) axis and the sympathetic nervous system (SNS) [15]. Since the stress response involves the release into blood of stress hormones such as cortisol, noradrenaline and adrenaline, their respective effects on the blood levels of glutamate and glucose were tested, the latter serving as a stress marker [16, 17].

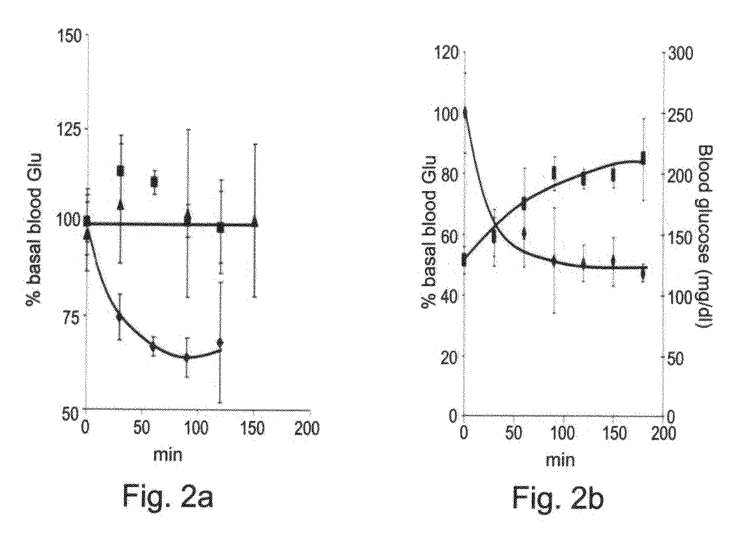

FIG. 2A illustrates the fact that while neither cortisol nor noradrenaline affected blood Glu levels, adrenaline, administered over 30 min, caused a sustained blood Glu decrease to about 60% of its basal levels. As excess brain Glu could cause the activation of a stress response [15], we tested the effects of the mere insertion into brain parenchyma of a microdialysis probe. FIG. 2B shows that the probe insertion is a stressful procedure as it caused a significant increase of blood glucose levels along with a concomitant decrease of blood Glu. These results suggest that adrenaline, released as part of the stress response to the insertion of the microdialysis probe, could be responsible for the spontaneous decrease of blood Glu levels observed in FIG. 1C.

### Example 3

### Stress Causes a Spontaneous Recovery from Traumatic Brain Injury

The decrease of blood Glu levels caused by stress was then addressed for its ability to neuroprotect from a brain insult well known to produce excess Glu levels in the brain of rodents [18-20] and of human victims of head injury [19, 21-23].

To this end, rats were subjected to a traumatic brain injury (TBI) [24], assessed a neurological severity score (NSS) 1, 24 and 48 h post injury, and monitored in parallel the blood Glu levels as described in FIG. 3A. The inflicted brain injury was very severe as expressed by the large NSS value of 16.2+/−0.5 (n=33) measured at 1 h post TBI (FIG. 3C-left first column). Animals appear nevertheless to rapidly recover since, in comparison to the NSS measured at 1 h post TBI, there was a very significant NSS improvement after 24 h (FIG. 3C-first two columns). As such spontaneous improvement could possibly be attributed to the stress/adrenaline-induced decrease of blood Glu levels. Therefore the effects of the non selective □-adrenergic blocking agent, propranolol was tested both on the NSS and on the blood Glu and glucose levels. FIG. 3B shows that a propranolol injection given 1 hour before TBI prevented the spontaneous NSS improvement normally observed after 24 h. It also prevented for at least 150 min the stress-induced changes of blood Glu and glucose levels (FIG. 3D). However, delaying the propranolol administration by 60 min after TBI (allowing thereby the TBI-related stress response) caused the same NSS improvement as that found after saline treatment (FIG. 3C). Thus, the spontaneous recovery after TBI is due to a stress-induced sympathetic discharge that causes a decrease of blood Glu. Incidentally, the propranolol pretreatment did not affect (data not shown) the Glu dual-probe microdialysis profile seen in FIG. 1A, suggesting that high Glu concentrations in brain are sufficient for a brain-to-blood Glu efflux, though the latter will be eased further by a reduction of blood Glu.

### Example 4

### Blood Glu Scavenging Improves and Accelerates Recovery from TBI

Thereafter the effect of increased brain-to-blood Glu efflux produced by scavenging blood Glu [10] on neuroprotection enhancement was addressed.

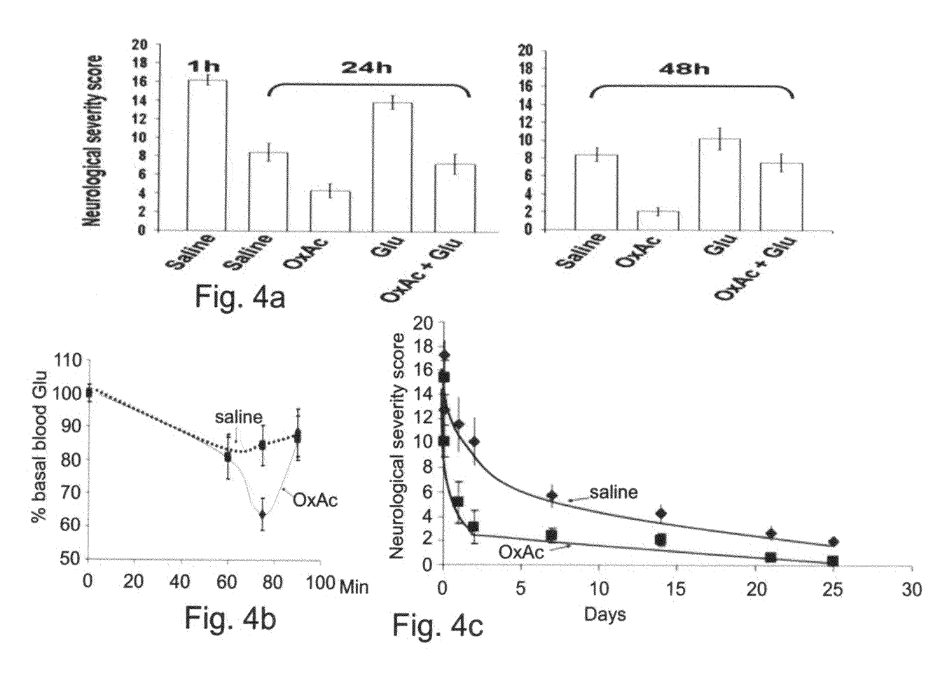

Therefore, rats were submitted to TBI and treated either with OxAc, Glu or saline, as described in FIG. 4A. The effects of a treatment with OxAc+Glu was also examined as the presence of Glu is expected to neutralize the OxAc-mediated decrease of blood Glu levels. FIG. 4A shows that animals treated with OxAc recovered best from TBI while those treated with Glu recovered the least. Animals treated with OxAc+Glu had a similar recovery as those treated with saline only.

To ascertain that OxAc caused a larger decrease of blood Glu levels than that caused by stress only, blood Glu levels were assayed before and after TBI, as well as the outcome of the subsequent 30 min-long treatments with either OxAc or saline. FIG. 4B shows that the stress produced by TBI reduced by 20% (Student t test p=5.10⁻⁴) the blood Glu levels measured after 60 min, and those were further reduced to 40% by the treatment with intravenous OxAc, but not with saline. The stress- and OxAc-induced decreases of blood Glu after TBI last for at least 90 min and are followed by a suggested compensatory Glu influx into blood from peripheral organs that sense the decrease in the blood Glu levels [10].

To compare the time course of the spontaneous recovery from TBI of saline-versus OxAc-treated rats, their NSS values were monitored for over 24 days. FIG. 4C shows that an extensive recovery from the neurological deficits inflicted by TBI occurs over this time period. However, the rate of recovery in the OxAc-treated rats is significantly faster than that of the saline-treated rats, and the latter remain at 25 days with a significant neurological handicap. These results strongly suggest that the spontaneous recovery of rats submitted to TBI includes a crucial step of elimination of excess brain Glu.

### Example 5

### The Recovery from TBI Correlates with the Decrease of Blood Glu Levels

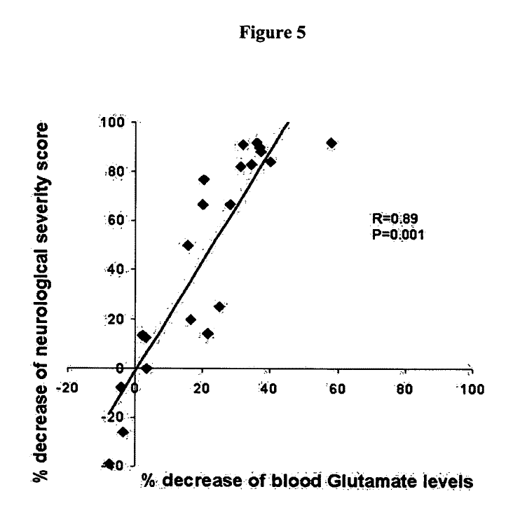

In order to demonstrate that the NSS changes are correlated with the changes of blood Glu levels, FIG. 5 was plotted in which the % NSS improvement of individual rats assessed 24 h after TBI versus the % decrease of their blood Glu levels measured 90 min after TBI are shown. A strong correlation (r=0.89) with a high statistical significance (p=0.001) was observed revealing that a 40% decrease of blood Glu levels affords an almost optimal improvement of the NSS. Thus, low blood Glu levels that facilitate an increased brain-to-blood Glu efflux appear to exert a brain neuroprotective effect while high blood Glu levels that prevent this brain self-protective process are deleterious. This conclusion is in line with the clinical observations that establish a linear correlation between CSF and plasma Glu concentrations [25], and a very significant association between the high blood Glu levels and the neurological deterioration and outcome after stroke [25, 26] or intracerebral hemorrhage [27].

To assess whether the treatment with OxAc improves, in addition to the NSS, other parameters that reflect the severity of the inflicted TBI, the extent of brain edema in OxAc-treated versus saline-treated animals were tested 120 min and 24 hours after TBI. Brain edema is partly due the presence of excess Glu since it is decreased by glutamate receptor antagonists [28-31]. The efficient removal of brain excess Glu is thus expected to decrease the edema.

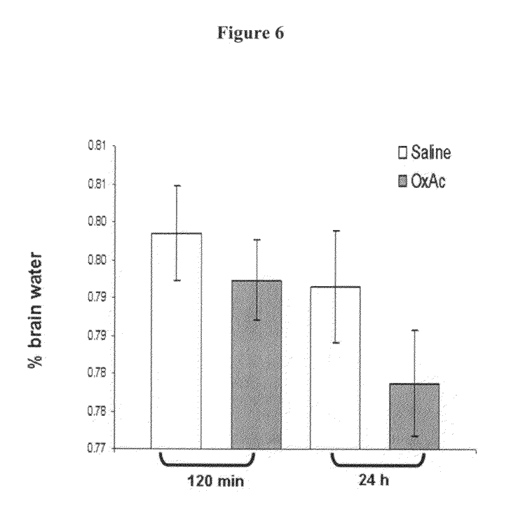

FIG. 6 shows that the OxAc-treatment caused a significant reduction of brain water content at 24 h. This beneficial effect was not observed in the other groups for which the treatment includes the intravenous administration of Glu (data not shown). Since OxAc could act on brain edema as an osmotic agent, both the blood osmolality and Na content were measured before and after the 30 min-long treatments with OxAc or saline. There was no change in the saline group (303±3.5 mOsm (pre-treatment) vs 299±3 mOsm (post-treatment); Na: 140±1 meq/l vs 141±0.8 meq/l) but a very significant increase in the OxAc-group: (301±5 mOsm vs 338±8 mOsm; Na: 139±1 meq/l vs 164±3.4 meq/l). The hypernatriema and hyperosmolarity in the OxAc-treated rats can be accounted by the fact that OxAc is administered together with 2 Na equivalents. To determine that the NSS improvement and edema reduction in the OxAc-treated rats were not due to an osmotic “therapy”, rats were submitted after TBI to a hypertonic (3% NaCl) saline treatment. As the respective NSS values obtained at 60 min and 24 h post TBI were 15.6±3.6 and 12.4±5.3 (n=7; p=0.16), it was concluded that the beneficial effects of OxAc are not the result of an osmotic therapy.

### Conclusions

Thus the above results demonstrate using dual probe microdialysis that a brain-to-blood efflux of excess Glu takes place from the parenchyma as it does from brain fluids [10]. This efflux is entirely regulated by the Glu concentration gradient between the ISF/capillary endothelial cell and blood plasma: it can be blocked by increasing blood Glu or be enhanced by the stress-induced activation of the sympathetic nervous system and/or by OxAc, a blood Glu scavenger. One may surmise that under normal conditions, the brain, via the Glu transporters on neurons and glia, has the means to take care very efficiently of local excess Glu, but it has to resort to a brain-to-blood Glu efflux, via Glu transporters on endothelial cells, in cases of large excess Glu that saturate the glial and neuronal transporters or of pathological conditions that impair their function. To assure the efficient removal of excess Glu, the brain activates a self protective decrease of blood Glu mediated by a stress hormone such as adrenaline. This phenomenon might possibly account for the rather limited duration of excess Glu in the rat brain [18, 20, 32] after TBI which lasts for about two hours, a time span fully compatible with the duration of the effects of adrenaline (see FIGS. 3A-C). As in man, excess Glu in brain can be observed after TBI for hours and even days [19, 21-23], one might conclude that a brain-to-blood Glu efflux is either not occurring or is poorly efficient. One might thus expect to improve it by the administration of blood Glu scavengers which may display here a therapeutic effect which has so far not been observed for glutamate receptor antagonists. One possible benefit is that the brain-to-blood Glu efflux is self limiting since it slows down in parallel with the decrease of brain Glu. Thus, it may not prevent Glu from exerting a role in neurorepair [33], a factor that has been suggested [34] to account for the failure of glutamate receptor antagonists in human clinical studies.

### Example 6

### Effect of Stress Hormone Agonists and Antagonists on Blood Glutamate Levels

Agents which qualify for reducing blood glutamate levels were assayed as follows.

Metaprolol (15 mg/kg) was injected into 4 naïve rats and the levels of blood Glu were determined at time=0 and every subsequent 30 min until 120 min. Rats (n=5) were preinjected with 15 mg/kg metaprolol and submitted to TBI. The rats NSS were measured after 24 and 48 h.

FIGS. 7A-B show the strong effect of a beta 1 antagonist, metaprolol on the reduction of blood Glu levels and NSS.

Phenylephrine was infused intravenously for 30 min into rats (n=4) at a rate 0.25 mg/0.5 ml/100 g/30 min and the levels of blood Glu were determined at time=0 and every subsequent 30 min until 120 min.

FIG. 8 is a graph showing the effect of an alpha 1 agonist, phenylephrine on blood Glu levels.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications mentioned in this specification are herein incorporated in their entirety by reference into the specification, to the same extent as if each individual publication, patent, patent application was specifically and individually indicated to be incorporated herein by reference. In addition, citation or identification of any reference in this application shall not be construed as an admission that such reference is available as prior art to the present invention.

### REFERENCES

### Other References are Cited in the Document

1. Castillo, J., et al., Neuroexcitatory amino acids and their relation to infarct size and neurological deficit in ischemic stroke. Stroke, 1996. 27(6): p. 1060-5.
2. Johnston, M. V., et al., Neurobiology of hypoxic-ischemic injury in the developing brain. Pediatr Res, 2001. 49(6): p. 735-41.
3. Zauner, A., et al., Glutamate release and cerebral blood flow after severe human head injury. Acta Neurochir Suppl, 1996. 67: p. 40-4.
4. Ferrarese, C., et al., Increased glutamate in CSF and plasma of patients with HIV dementia. Neurology, 2001. 57(4): p. 671-5.
5. Shaw, P. J., et al., CSF and plasma amino acid levels in motor neuron disease. elevation of CSF glutamate in a subset of patients. Neurodegeneration, 1995. 4(2): p. 209-16.
6. Spranger, M., et al., Excess glutamate in the cerebrospinal fluid in bacterial meningitis. J Neurol Sci, 1996. 143(1-2): p. 126-31.
7. Spreux-Varoquaux, O., et al., Glutamate levels in cerebrospinal fluid in amyotrophic lateral sclerosis: a reappraisal using a new HPLC method with coulometric detection in a large cohort of patients. J Neurol Sci, 2002. 193(2): p. 73-8.
8. Danbolt, N. C., Glutamate uptake. Prog Neurobiol, 2001. 65(1): p. 1-105.
9. Sattler, R. and M. Tymianski, Molecular mechanisms of glutamate receptor-mediated excitotoxic neuronal cell death. Mol Neurobiol, 2001. 24(1-3): p. 107-29.
10. Gottlieb, M., Y. Wang, and V. I. Teichberg, Blood-mediated scavenging of cerebrospinal fluid glutamate. J Neurochem, 2003. 87(1): p. 119-26.
11. O'Kane, R. L., et al., Na(+)-dependent glutamate transporters (EAAT1, EAAT2, and EAAT3) of the blood-brain barrier. A mechanism for glutamate removal. J Biol Chem, 1999. 274(45): p. 31891-5.
12. Hoistad, M., et al., Intracerebral infusion of H-dopamine and H-mannitol in the striatum of halothane-anaesthetized male rats. A dual-probe microdialysis study of long-distance diffusion. Eur J Neurosci, 2000. 12(7): p. 2505-14.
13. Benveniste, H. and P. C. Huttemeier, Microdialysis—theory and application. Prog Neurobiol, 1990. 35(3): p. 195-215.
14. Binder, D. K., et al., In vivo measurement of brain extracellular space diffusion by cortical surface photobleaching. J Neurosci, 2004. 24(37): p. 8049-56.
15. Zelena, D., Z. Mergl, and G. B. Makara, Glutamate agonists activate the hypothalamic-pituitary-adrenal axis through hypothalamic paraventricular nucleus but not through vasopressinerg neurons. Brain Res, 2005. 1031(2): p. 185-93.
16. Miller, D. B. and J. P. O'Callaghan, Neuroendocrine aspects of the response to stress. Metabolism, 2002. 51(6 Suppl 1): p. 5-10.
17. Rovlias, A. and S. Kotsou, The influence of hyperglycemia on neurological outcome in patients with severe head injury. Neurosurgery, 2000. 46(2): p. 335-42; discussion 342-3.
18. Palmer, A. M., et al., Traumatic brain injury-induced excitotoxicity assessed in a controlled cortical impact model. J Neurochem, 1993. 61(6): p. 2015-24.
19. Richards, D. A., et al., Extracellular glutamine to glutamate ratio may predict outcome in the injured brain: a clinical microdialysis study in children. Pharmacol Res, 2003. 48(1): p. 101-9.
20. Rose, M. E., et al., Regulation of interstitial excitatory amino acid concentrations after cortical contusion injury. Brain Res, 2002. 943(1): p. 15-22.
21. Baker, A. J., et al., Excitatory amino acids in cerebrospinal fluid following traumatic brain injury in humans. J Neurosurg, 1993. 79(3): p. 369-72.
22. Bullock, R., et al., Factors affecting excitatory amino acid release following severe human head injury. J Neurosurg, 1998. 89(4): p. 507-18.
23. Vespa, P., et al., Increase in extracellular glutamate caused by reduced cerebral perfusion pressure and seizures after human traumatic brain injury: a microdialysis study. J Neurosurg, 1998. 89(6): p. 971-82.
24. Shapira, Y., et al., Experimental closed head injury in rats: mechanical, pathophysiologic, and neurologic properties. Crit Care Med, 1988. 16(3): p. 258-65.
25. Castillo, J., A. Davalos, and M. Noya, Progression of ischaemic stroke and excitotoxic aminoacids. Lancet, 1997. 349(9045): p. 79-83.
26. Serena, J., et al., Neurological deterioration in acute lacunar infarctions: the role of excitatory and inhibitory neurotransmitters. Stroke, 2001. 32(5): p. 1154-61.
27. Castillo, J., et al., Molecular signatures of brain injury after intracerebral hemorrhage. Neurology, 2002. 58(4): p. 624-9.
28. Furukawa, T., et al., The glutamate AMPA receptor antagonist, YM872, attenuates cortical tissue loss, regional cerebral edema, and neurological motor deficits after experimental brain injury in rats. J Neurotrauma, 2003. 20(3): p. 269-78.
29. Shapira, Y., A. A. Artru, and A. M. Lam, Ketamine decreases cerebral infarct volume and improves neurological outcome following experimental head trauma in rats. J Neurosurg Anesthesiol, 1992. 4(4): p. 231-40.
30. Westergren, I. and B. B. Johansson, NBQX, an AMPA antagonist, reduces glutamate-mediated brain edema. Brain Res, 1992. 573(2): p. 324-6.
31. Westergren, I. and B. B. Johansson, Blockade of AMPA receptors reduces brain edema following opening of the blood-brain barrier. J Cereb Blood Flow Metab, 1993. 13(4): p. 603-8.
32. Faden, A. I., et al., The role of excitatory amino acids and NMDA receptors in traumatic brain injury. Science, 1989. 244(4906): p. 798-800.
33. Biegon, A., et al., Dynamic changes in N-methyl-D-aspartate receptors after closed head injury in mice: Implications for treatment of neurological and cognitive deficits. Proc Natl Acad Sci USA, 2004. 101(14): p. 5117-22.
34. Ikonomidou, C. and L. Turski, Why did NMDA receptor antagonists fail clinical trials for stroke and traumatic brain injury? Lancet Neurol, 2002. 1(6): p. 383-6.
35. Graham, L. T., Jr. and M. H. Aprison, Fluorometric determination of aspartate, glutamate, and gamma-aminobutyrate in nerve tissue using enzymic methods. Anal Biochem, 1966. 15(3): p. 487-97.

1. A method of reducing extracellular brain glutamate levels, the method comprising administering to a subject in need thereof an agent capable of modulating stress hormone activity thereby reducing blood glutamate levels, thereby reducing extracellular brain glutamate levels.

2. A pharmaceutical composition comprising as active ingredients at least two agents capable of reducing blood glutamate levels, wherein at least one of said at least two agents is capable of modulating stress hormone activity thereby reducing blood glutamate levels and a pharmaceutically acceptable carrier.

3. (canceled)

4. A method of reducing extracellular brain glutamate levels in a subject in need thereof, the method comprising:
(a) obtaining a blood sample;
(b) contacting said blood sample with an agent capable of modulating stress hormone activity thereby reducing glutamate levels of cells present in said blood sample to thereby obtain glutamate depleted blood cells; and
(c) introducing said glutamate depleted blood cells into the subject, thereby reducing extracellular brain glutamate levels thereof.

5. The methods, of claim 1, wherein said agent capable of modulating stress hormone activity thereby reducing blood glutamate levels is a stress hormone agonist.

6. The methods, claim 1, wherein said agent capable of modulating stress hormone activity thereby reducing blood glutamate levels is a stress hormone antagonist.

7. The method of claim 5, wherein said stress hormone agonist comprises an adrenergic receptor agonist.

8. The method of claim 7, wherein said adrenergic receptor agonist is an alpha 1 or alpha 2 agonist.

9. The method of claim 7, wherein said adrenergic receptor agonist is a beta 2 agonist.

10. The method of claim 6, wherein said stress hormone antagonist comprises is an adrenergic receptor antagonist.

11. The method of claim 10, wherein said adrenergic receptor antagonist is a beta 1 antagonist.

12. The method of claim 1 further comprising administering an additional agent capable of reducing blood glutamate levels prior to, concomitant with or following administering said stress hormone.

13. (canceled)

14. The method of claim 12, wherein said additional agent is at least one glutamate modifying enzyme and/or a modification thereof.

15. The method of claim 14, wherein said at least one glutamate modifying enzyme is selected from the group consisting of a transaminase, a dehydrogenase, a decarboxylase, a ligase, an aminomutase, a racemase and a transferase.

16. The method of claim 15, wherein said transaminase is selected from the group consisting of glutamate oxaloacetate transaminase, glutamate pyruvate transaminase, acetylornithine transaminase, ornithine-oxo-acid transaminase, succinyldiaminopimelate transaminase, 4-aminobutyrate transaminase, alanine transaminase, (s)-3-amino-2-methylpropionate transaminase, 4-hydroxyglutamate transaminase, diiodotyrosine transaminase, thyroid-hormone transaminase, tryptophan transaminase, diamine transaminase, cysteine transaminase, L-Lysine 6-transaminase, histidine transaminase, 2-aminoadipate transaminase, glycine transaminase, branched-chain-amino-acid transaminase, 5-aminovalerate transaminase, dihydroxyphenylalanine transaminase, tyrosine transaminase, phosphoserine transaminase, taurine transaminase, aromatic-amino-acid transaminase, aromatic-amino-acid-glyoxylate transaminase, leucine transaminase, 2-aminohexanoate transaminase, ornithine(lysine) transaminase, kynurenine-oxoglutarate transaminase, D-4-hydroxyphenylglycine transaminase, cysteine-conjugate transaminase, 2,5-diaminovalerate transaminase, histidinol-phosphate transaminase, diaminobutyrate-2-oxoglutarate transaminase, udp-2-acetamido-4-amino-2,4,6-trideoxyglucose transaminase and aspartate transaminase.

17. The method of claim 15, wherein said dehydrogenase is a glutamate dehydrogenase.

18. The method of claim 15, wherein said decarboxylase is a glutamate decarboxylase.

19. The method of claim 15, wherein said ligase is a glutamate-ethylamine ligase.

20. The method of claim 15, wherein said transferase is selected from the group consisting of glutamate n-acetyltransferase and adenylyltransferase.

21. The methods and pharmaceutical composition of claim 15, wherein said aminomutase is a glutamate-1-semialdehyde 2,1-aminomutase.

22. The method of claim 12, wherein said additional agent is at least one co-factor of a glutamate modifying enzyme.

23. The method of claim 22, wherein said co-factor is selected from the group consisting of oxaloacetate, pyruvate, NAD⁺, NADP⁺, 2-oxohexanedioic acid, 2-oxo-3-sulfopropionate, 2-oxo-3-sulfinopropionic acid, 2-oxo-3-phenylpropionic acid, 3-indole-2-oxopropionic acid, 3-(4-hydroxyphenyl)-2-oxopropionic acid, 4-methylsulfonyl-2-oxobutyric acid, 3-hydroxy-2-oxopropionic acid, 5-oxopentanoate, 6-oxo-hexanoate, glyoxalate, 4-oxobutanoate, α-ketoisocaproate, α-ketoisovalerate, α-keto-β-methylvalerate, succinic semialdehyde-(-4-oxobutyrate), pyridoxal phosphate, pyridoxal phosphate precursor and 3-oxoisobutanoate.

24-27. (canceled)

28. The method of claim 12, wherein said additional agent includes a glutamate modifying enzyme and a co-factor thereof.

29-41. (canceled)
